(19) United States
(12) Patent Application Publication
(10) Pub. No.: US 2023/0302145 A1
(43) Pub. Date: Sep. 28, 2023

(54) SURFACTANT PROTEIN C MIMICS DISPLAYING PATHOGEN- OR ALLERGEN-BINDING MOIETIES

(71) Applicant: MAXWELL BIOSCIENCES, INC., Austin, TX (US)

(72) Inventors: ANNELISE E. BARRON, Woodside, CA (US); JOHN A. FORTKORT, Austin, TX (US)

(21) Appl. No.: 17/998,337

(22) PCT Filed: May 10, 2021
(86) PCT No.: PCT/US2021/031663

§ 371 Date: Jun. 9, 2023

Related U.S. Application Data
(60) Provisional application No. 63/022,572, filed on May 10, 2020.

(51) Int. Cl. A61K 47/64; A61P 31/14

(52) U.S. Cl. CPC A61K 47/64; A61P 31/14

(57) ABSTRACT

A method is provided for treating a subject. The method comprises diagnosing the subject as suffering from a condition arising from the presence in the subject of a causative agent; and administering to the subject a pharmaceutically effective amount of a substance having (a) a hydrophobic, helical region, (b) an N-terminal region that includes at least one proline residue, (c) a first linking moiety that links the hydrophobic helical region to the N-terminal region, said linking moiety being equipped with at least one lysine-like side chain, (d) a binding moiety which binds to the causative agent, and (e) a second linking moiety that links the binding moiety to the N-terminal region.

### CROSS-REFERENCE TO RELATED APPLICATION

This application claims the benefit of priority of U.S. Pat. Application No. 63/022,572, filed May 10, 2020, having the same inventors and title, which is incorporated herein by reference in its entirety.

### FIELD OF THE DISCLOSURE

The present disclosure relates generally to methods for the treatment and prevention of diseases, and more particularly to methods for treating and preventing infections or allergic responses through the use of pulmonary surfactants equipped with binding moieties for pathogens, allergens or other infectious agents.

### BACKGROUND OF THE DISCLOSURE

Pulmonary surfactant (PS) is a surface-active lipoprotein complex which is produced by type II alveolar cells and which is essential for normal respiration. Human PS consists of approximately 10% proteins and 90% lipids. The protein portion includes four proteins: (1) the collectins SP-A and SP-D, which are water-soluble, innate immune proteins; and (2) the hydrophobic, surface-active surfactant proteins, SP-B and SP-C, which are critical for the biophysical function of PS to reduce the work of breathing (in particular, these membrane proteins increase the rate at which PS spreads over the surface of the lungs). The lipid portion of PS consists of a mixture of many different lipids, as well as cholesterol. Among these are saturated phospholipids, about 85% of which are phosphatidylcholines. The primary phosphatidylcholine in PS, which is most critical for surface tension reduction, is dipalmitoyl phosphatidylcholine (DPPC).

The constituent proteins and lipids of PS have both hydrophilic and hydrophobic regions. In the airways, PS functions to (1) form an interfacial surfactant layer by rapidly adsorbing to the alveolar air-liquid interface; (2) prevent alveolar collapse by greatly reducing the interfacial surface tension upon compression to near zero values (expiration); and (3) reduce the maximum surface tension (and diminish the effort required to breath) by efficiently respreading upon expansion (inhalation).

DPPC and other phospholipids reduce surface tension in PS by forming a mixed lipid monolayer / bilayer / multilayer at the air-water interface of alveoli, such that the hydrophilic head groups of DPPC are disposed in the water portion, and the hydrophobic tails of DPPC are facing the air side. The biophysical properties that allow saturated phospholipids to achieve very low surface tensions also prevent these lipids from rapidly reabsorbing and respreading upon expansion. The addition of hydrophobic proteins SP-B and SP-C to the lipid portion greatly enhances surfactant adsorption, stability, and recycling of the lipid film. The inclusion of these surfactant-specific proteins is therefore necessary for proper respiration, and their omission can result in potentially lethal respiratory failure.

Several human lung diseases, such as those that can occur in premature babies with immature lungs, result from deficiencies in pulmonary surfactant material. These diseases are commonly treated with the application of exogenous lung surfactant preparations, which have historically been derived from animal-sourced materials.

Although natural, animal-derived surfactant preparations have proven to be efficacious in many applications, the use of these materials has some notable drawbacks. In particular, the use of animal-sourced materials creates the potential for cross-species transfer of infectious agents, has high production costs, and is subject to batch-to-batch variabilities in the composition of the surfactant materials.

There has thus been considerable effort in the art to develop synthetic PS formulations. This effort has led to the development of synthetic pulmonary surfactants. Thus, for example, U.S. 10,532,066 (Voelker et al.), entitled “Surfactant Lipids, Compositions Thereof, And Uses Thereof”, discloses anionic lipids having (a) has a hydrophobic portion, (b) a negatively charged portion, and (c) an uncharged, polar portion. U.S. 2020/0009165 (Voelker), entitled “Methods And Compositions For Treating And Preventing Respiratory Related Diseases And Conditions With Xylitol-Headgroup Lipid Analogs”, discloses xylitol lipid analogs having (a) a phospholipid glycerol backbone, (b) a xylitol polar headgroup, (c) a phosphodiester bond linking the glycerol backbone to the xylitol polar headgroup, and (d) variable hydrophobic regions comprising two aliphatic chains of 14 to 18 carbons in length, wherein linkage between the aliphatic chains and the phospholipid glycerol backbone is an O-acyl linkage or an O-alkyl linkage; and the aliphatic chains contain 0 to 2 double bonds.

Synthetic analogs of PS proteins have also been developed. For example, Brown, Nathan & Lin, Jennifer & Barron, Annelise. (2019), “Helical Side Chain Chemistry of a Peptoid-Based SP-C Analogue: Balancing Structural Rigidity And Biomimicry”, Biopolymers. 110. 10.1002/bip.23277 (Brown et al.) discloses synthetic non-natural mimics of SP-C using a poly-N-substituted glycine (or “peptoid”) backbone. Brown et al. cites prior literature in which earlier generations of peptoid mimics of SP-C were reported and also discusses related work.

### SUMMARY OF THE DISCLOSURE

In one aspect, a method is provided for treating a subject. The method comprises diagnosing the subject as suffering from a condition arising from the presence in the subject of a causative agent; and administering to the subject a pharmaceutically effective amount of a substance having (a) a hydrophobic, helical region, (b) an N-terminal region that includes at least one proline residue, (c) a first linking moiety that links the hydrophobic helical region to the N-terminal region, said linking moiety being equipped with at least one lysine-like or arginine-like side chain, (d) a binding moiety which binds to the causative agent, and (e) a second linking moiety that links the binding moiety to the N-terminal region.

In another aspect, a method for treating an infection caused by a pathogen is provided. The method comprises administering to an individual who has said infection or who is at risk of developing said infection, an amount of at least one composition, wherein the amount of the composition is effective to inhibit said infection, wherein the composition includes (a) a pulmonary surfactant protein mimic, which is either a polypeptide or a polypeptoid (poly-N-substituted glycine), (b) a binding moiety which binds to said pathogen, and (c) a linking moiety that links the binding moiety to the pulmonary surfactant protein mimic.

In a further aspect, a method is provided for treating an allergy caused by an associated allergen. The method comprises administering to an individual who has said allergy an amount of at least one composition, wherein the amount of the composition is effective to inhibit said allergy, and wherein the composition includes (a) a pulmonary surfactant protein mimic, which is either a polypeptide or a polypeptoid (poly-N-substituted glycine), (b) a binding moiety which binds to said allergen, and (c) a linking moiety that links the binding moiety to the pulmonary surfactant protein mimic.

In still another aspect, a method for treating an individual for an infection caused by a pathogen is provided. The method comprises administering to an individual who has said infection or who is at risk of developing said infection, a pharmaceutically effective amount of at least one composition, wherein the amount of the composition is effective to inhibit said infection, and wherein the composition includes a binder which binds to the pathogen, wherein said binder is a peptide or peptoid mimic of a receptor that the pathogen binds to.

### BRIEF DESCRIPTION OF THE DRAWINGS

FIG. 1 is an illustration of the molecular structures of a set of peptoid mimics of Surfactant Protein C (SP-C) which may be utilized as precursors in making binding mimics in accordance with the teachings herein. Abbreviated names for the depicted set of peptoid monomers are also shown.

FIG. 2 is an illustration of the varied helical chemistry of a library of different peptoid mimics of SP-C, some of which are depicted in FIG. 1.

FIG. 3 is an illustration of further, various peptoid mimics of SP-C, some of which are depicted in FIG. 1, and some of which are more biomimetic by virtue of a different molecular design for the monomer used in the helical region.

FIG. 4 is a listing of the N-substituted glycine monomer sequences of some of the precursor peptoid mimics which can be utilized in making binding mimics in accordance with the teachings herein.

FIG. 5 is an illustration of the molecular structures of a set of binding mimics in accordance with the teachings herein.

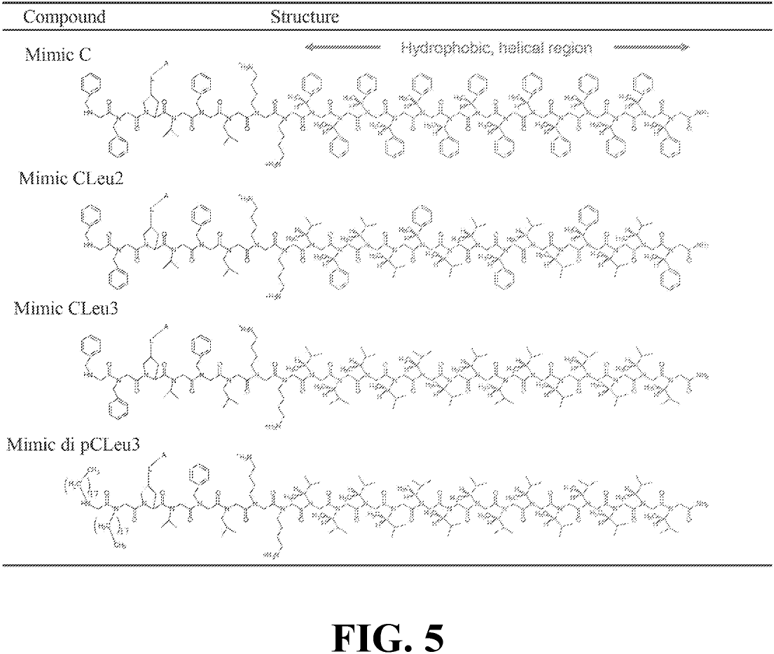

FIG. 6 is an illustration of the molecular structure of a set of peptoid mimics of SP-C which may be utilized as precursors in making binding mimics in accordance with the teachings herein, and which shows the peptoid helix length and side chain chemistry of the precursors.

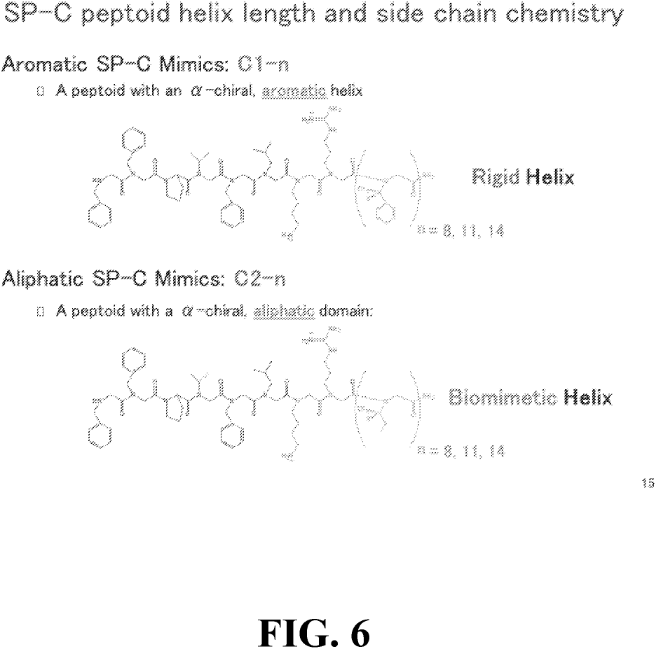

### DETAILED DESCRIPTION

While some of the synthetic lung surfactants produced to date may have many desirable attributes, further improvements in these materials are desired. By way of example, some diseases, such as coronavirus infections (including, for example, COVID-19 which is caused by the SARS-CoV-2 coronavirus; severe acute respiratory syndrome (SARS) which is caused by the SARS-CoV or SARS-CoV-1 coronavirus; and Middle East respiratory syndrome (MERS) which is caused by the MERS-coronavirus (MERS-CoV)) may induce or involve deficiencies in, or damage to, pulmonary surfactant (PS) material in the lungs of patients. This condition may allow the associated pathogen (here, a coronavirus) to invade the pulmonary epithelial cells, which may lead to worsening symptoms in the victim and progression of the disease. Although supplementing the native PS with a synthetic, exogenous, biomimetic surfactant may help lessen the severity of symptoms in some cases, such an approach does not necessarily treat the causative agent and may therefore fail to resolve the patient’s condition.

It has now been found that the foregoing issue may be addressed with the class of novel synthetic pulmonary surfactants disclosed herein. In a preferred embodiment, these pulmonary surfactants, which are referred to herein as “binding mimics”, include a base to which a binding moiety is attached, preferably via a linking moiety. The base is preferably a PS protein or PS protein mimic (which is preferably based upon a poly-N-substituted glycine or “peptoid” structure, but could also be a polypeptide mimic of the PS protein), and is more preferably an SP-C mimic, although various embodiments of binding mimics are possible in accordance with the teachings herein in which the base is (or is a mimic of) other surfactant proteins (including surfactant proteins SP-A, SP-B and SP-D), lipids (including, for example, cholesterol), phospholipids (including, for example, dipalmitoylphosphatidylcholine (DPPC)) or phosphotides.

The binding moiety is preferably selected to bind to one or more pathogens or other causative or infectious agents of interest. Without wishing to be bound by theory, these binding mimics are believed to act by binding to such causative agents, thus deactivating or immobilizing them until they (or the resulting reaction product) can be destroyed or removed from the body by the native immune system or other natural processes.

The linking moiety is preferably selected to provide suitable spacing between the base and the binding moiety, and in some cases may also impart desirable rotational or orientational characteristics to the binding mimic. The linking moiety may also be sufficiently labile or prone to decomposition (for example, by undergoing proteolysis, in the event that the linking moiety is a protein or comprises amino acid sequences).

By way of example but not limitation, within the lungs, the binding mimic may be utilized to bind and immobilize, deactivate, destroy or remove pathogens, allergens and other causative agents, infectious agents or targets of interest. Since the binding mimic includes a base (which may be, for example, an SP-C mimic) which may act to anchor the molecule to the bilipid layer typically present in the lungs, this may have the effect of binding the target of interest to the bilipid layer until it can be deactivated, destroyed or removed from the body by the native immune system or by other natural processes.

In the case of the SARS-CoV-2 coronavirus, for example, the binding mimic may be equipped with a binding moiety (such as, for example, a mimic of part of the ACE-2 receptor) that binds to the virus (or to which the virus binds), thus causing the viral particles to remain adhered to the external surfactant bilipid layer (by way of binding to the binding moiety, which is attached to an SP-C mimic) and preventing them from entering the lung epithelial cells and causing infection. The bound viral particles may then be removed from the lungs through, for example, the normal cleansing action of the cilia or by other native processes. It will be appreciated that this approach has both prophylactic and therapeutic applications, since both the prevention and treatment of diseases such as COVID-19 are reliant upon arresting the spread of the causative pathogen, and in particular, preventing the entry of the viral particles into host lung epithelial cells.

In especially preferred embodiments, the binding mimics disclosed herein may include (a) a hydrophobic, helical region, (b) an N-terminal region that includes at least one proline residue, (c) a first linking moiety that links the hydrophobic helical region to the N-terminal region, said first linking moiety being equipped with at least one lysine-like side chain and/or one arginine-like side chain, (d) a binding moiety which is selected to bind to pathogens or agents of interest, and (e) a second linking moiety which is preferably a short, water-soluble and flexible linking moiety that links the binding moiety to the N-terminal region.

The binding mimics disclosed herein are preferably derived through suitable modification of pulmonary surfactant mimics (and more specifically, mimics of pulmonary surfactant proteins). These pulmonary surfactant mimics will most preferably be mimics of human SP-C or portions thereof, although mimics of other pulmonary surfactant proteins (or portions thereof) are also possible. These include, without limitation, mimics of SP-A, SP-B or SP-D (or portions thereof), and mimics of surfactant proteins (or portions thereof) from other species such as, for example, porcine versions of SP-C. The modifications to these pulmonary surfactant mimics will preferably involve installation of one or more binding moieties on the mimic (preferably via one or more suitable linking moieties), thus yielding a binding mimic. The binding moieties and linking moieties are preferably selected to maintain the stability and conformational state, 3-D structure and general functional properties of the underlying mimic such that the resulting binding mimic continues to function as a pulmonary surfactant mimic.

In some embodiments, the binding mimic may feature a base which is, or is a mimic of, a lipid (including, for example, cholesterol), a phospholipid (including, for example, dipalmitoylphosphatidylcholine (DPPC)) or another suitable phospholipid.

As previously noted, in a preferred embodiment, the binding mimics disclosed herein are preferably derived from SP-C mimics. SP-C is a helical, extraordinarily hydrophobic protein which is 35-amino acids in length. It is highly sequence-conserved among all mammalian species. SP-C contains a 37-Å-long helical region. This helical region is capable of traversing the lipid bilayer, and associates (and interacts with) the interior of phospholipid acyl chains. In addition, the N-terminal region of natural human SP-C contains two palmitoylated cysteines at positions 5 and 6. The two palmitoyl chains are believed to play a key role in maintaining the association between SP-C and associated phospholipids within the interfacial surfactant film at very high levels of compression. Hence, the palmitoylated cysteines act as a hydrophobic “anchor” for the excluded surfactant material and aid in the reincorporation of this material during expansion. Palmitoylation of these cysteines has also been shown to be vital in maintaining the rigid α-helical structure of SP-C.

The important biophysical activities of natural SP-C protein, and its inclusion in animal-derived surfactants, underscore the critical role of this protein in pulmonary surfactant compositions. Unfortunately, large-scale production of this protein is exceedingly difficult (due, in part, to its highly hydrophobic nature), thus making its incorporation into synthetic surfactant preparations impractical. Moreover, although SP-C is relatively small and lacks any tertiary structure, the native protein and its sequence-identical analogues are challenging to work with. For example, the poly-valyl helix is composed entirely of aliphatic residues with β-branched side chains which, in the absence of lipids, spontaneously convert into β-sheet aggregate structures with reduced surface activity.

The difficulties associated with native SP-C (and in particular, its metastable secondary structure and aggregation propensity) have been overcome through the use of SP-C mimics. In designing appropriate peptidomimetics, consideration should be given to the characteristics that are essential to create more manageable SP-C analogues that retain the key functionality of SP-C. Accordingly, structure-function studies have been undertaken on SP-C which have revealed some of the molecular features that are preferably retained in order to retain the protein’s functionality. These studies have underscored the desirability of retaining the protein’s extreme hydrophobicity, duplication of its longitudinally amphipathic patterning of hydrophobic and polar residues, and maintenance of its rigid, helical secondary structure.

Many of the surface-active properties of SP-C are known to be facilitated by the protein’s valyl-rich helical region, the length of which approximates the thickness of a DPPC bilayer (37 Å). It has been found that the α-helical conformation and overall hydrophobicity is of greater importance than the exact side chain chemistry in capturing SP-C′s surface-active properties, thus opening up the possibility of preserving the desired SP-C molecular parameters in peptidomimetics with alternative (yet still hydrophobic) side-chain structures. This approach may be utilized to simplify the production and handling of SP-C analogues.

Poly-N-substituted glycines (or “peptoids”) may be utilized to mimic SP-C, and are a preferred class of mimics for use in the fabrication of the binding mimics disclosed herein. Peptoids are structurally similar to peptides, being based on an similar backbone structures, except that the side chains are attached to the amide nitrogens rather than to the α-carbons of the constituent amino acids. Peptoids are resistant to protease degradation and are more biostable than peptides as a result of this modified positioning of the side chains. Compared to peptides, peptoids are also relatively simple and cost-effective to synthesize, although the methods of solid-phase synthesis are largely similar.

Unlike polypeptides, the unsubstituted methylene carbon of the peptoid backbone is achiral. Moreover, because the backbone nitrogens are substituted with side chains, peptoids also lack backbone hydrogen bond donors. Despite this, peptoids with α-chiral, sterically bulky side chains are capable of assuming extraordinarily stable, handed helices. Consequently, peptoids are excellent candidates for mimicking bioactive molecules (such as the hydrophobic proteins of pulmonary surfactants) that rely on helical structures to function properly. These helical structures are physically similar in structure to a polyproline type I helix, and have about 3 residues per turn with a helical pitch of about 6 Å. Notably, many of the same design strategies used in the development of SP-C peptide-based analogues are also applicable to peptoid-based analogues and binding mimics based on the same. Similar to the peptide-based analogues, peptoid-based analogues containing more rigid helices display superior SP-C-like behaviors in comparison to peptoid-based analogues containing more flexible aliphatic-based helices. This suggests that the overall secondary structure and hydrophobicity of SP-C are the more important features to mimic, rather than the exact side chain chemistries.

Despite the superior surface activity of the more rigid (aromatic side chain-based) peptoid mimics of SP-C, the aliphatic-based mimics display some desirable properties, thus making them the materials of choice as precursors for some applications of the binding mimics disclosed herein. Such properties include a lower maximum surface tension during dynamic cycling, which indicates favorable interactions between the branched aliphatic side chains and the lipid acyl chains. The preservation of these interactions may be functionally important in light of the fact that the SP-C poly-valyl helix is typically conserved, although it is presently unclear whether this is simply an adaption to the extremely hydrophobic lipid environment, or is one of functional necessity.

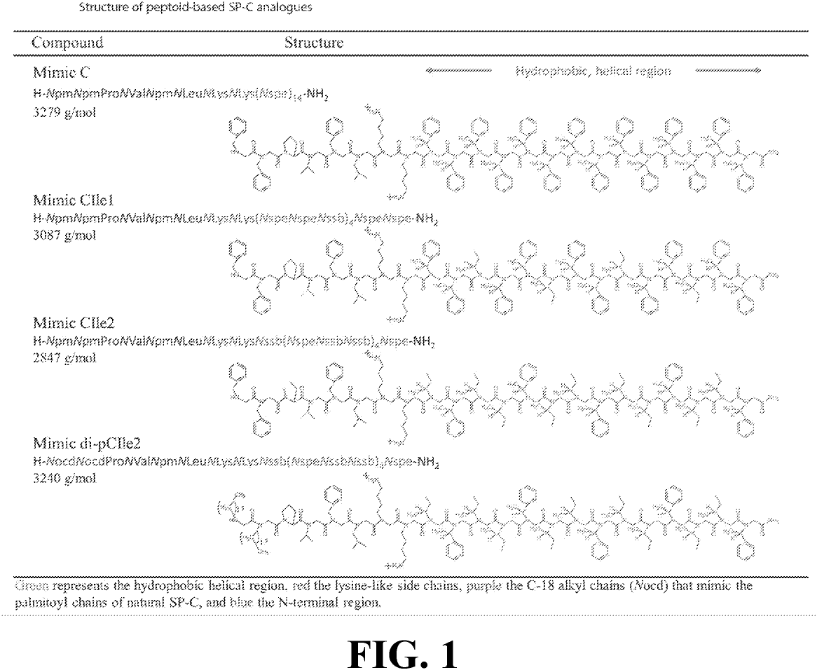

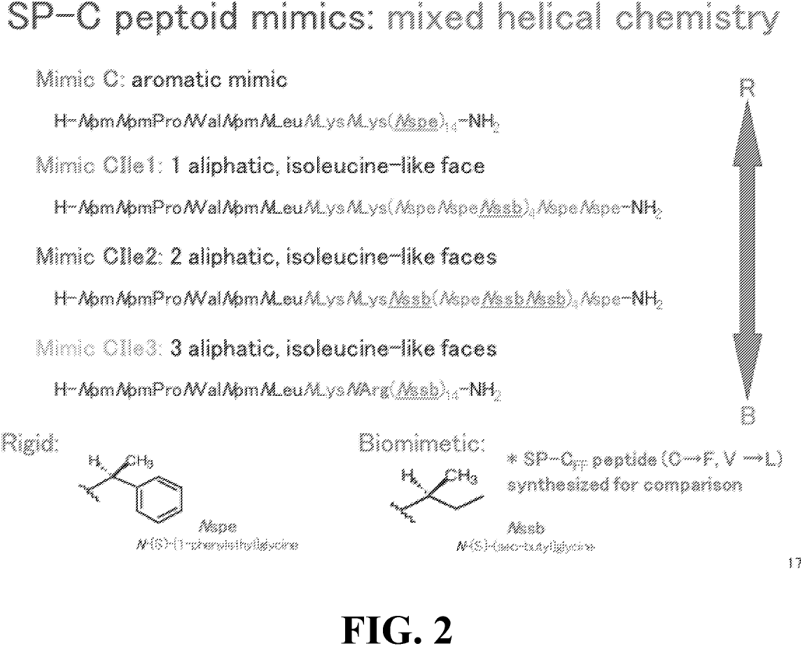

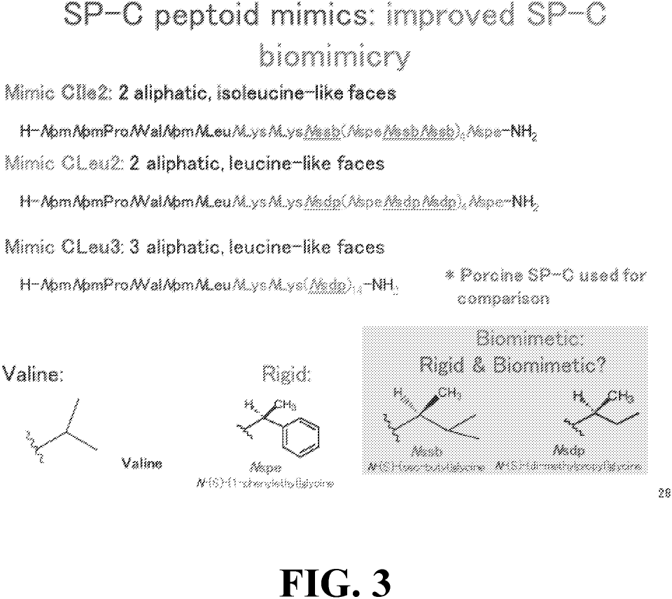

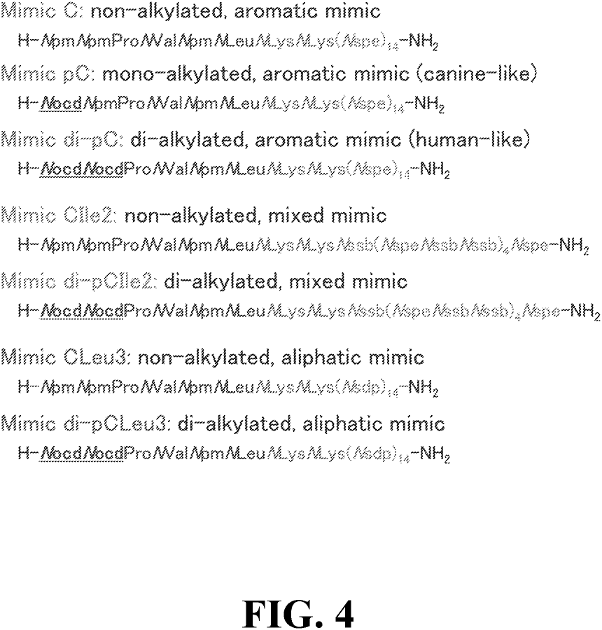

In light of the foregoing, a set of peptoid-based mimics have been created to serve as precursors for the binding mimics disclosed herein. These precursors, which are depicted in FIG. 1, were produced and characterized with the intention of optimizing the molecular features of the SP-C mimics by incorporating both α-chiral, aromatic side chains and α-chiral, aliphatic side chains. FIGS. 2-4 depict some of the characteristics of these precursors. Preferably, the linking moieties and binding moieties utilized to produce binding mimics from these precursors are selected to preserve the characteristics of the underlying precursors.

In preferred embodiments, the designed binding mimics contain varying amounts of aromatic and aliphatic residues in the 14-residue helical region (i.e., all-aromatic, 10 aromatic/4 aliphatic, and 5 aromatic/9 aliphatic side chains). This approach permits imparting two molecular characteristics to one binding mimic by obtaining structural rigidity from the aromatic side chains and side chain biomimicry (i.e., mimicking valine structure) from the aliphatic side chains. It has been found that increasing the aliphatic content in the helical region incrementally increases the in vitro surface activity of the precursor, causing a reduction in maximum surface tension during dynamic cycling. The extents of rigidity and biomimicry may be balanced and optimized through the incorporation into the helical region of approximately one-third aromatic side chains for structural rigidity and two thirds aliphatic side chains for side chain biomimicry. This process results in a set of precursor mimics that display better surface activity in many applications than precursor mimics composed solely of either aromatic or aliphatic side chains.

To further improve the surface activity of some of the most promising binding mimics, two alkyl chains may be introduced in the N-terminal region. The amide-linked C-18 alkyl chains mimic the structure and hydrophobicity of the palmitoyl chains of SP-C that are responsible for important surface-active properties, and are stable at the point of linkage. Alkylation may further improve the surface activity of these binding mimics, resulting in a surfactant film with comparable in vitro surface activities to a natural SP-C-containing formulation.

It will be appreciated from the foregoing that various peptoid mimics may be utilized as precursors to the binding mimics disclosed herein. Preferred precursors include the mimics denoted as CLeu3 and di-pCLeu3 herein, with the precursor denoted as mono-pCLeu3 (this precursor has just one octadecyl modification at position 1 in the sequence) being especially preferred.

The following, non-limiting examples illustrate various aspects of the compositions and methodologies described herein.

### Example 1

This example illustrates the synthesis of peptoid mimic precursors utilized in making binding mimics of the type disclosed herein.

The peptoid-based SP-C mimics of FIG. 1 were synthesized on an automated 433A ABI Peptide Synthesizer (Foster City, CA) on solid support (Rink amide resin), following a two-step submonomer method as described by Zuckermann et al. [R. N. Zuckermann, J. M. Kerr, S. B. H. Kent, W. H. Moos, J. Am. Chem. Soc. 1992, 114(26), 10646]. Briefly, synthesis was carried out on 0.25 mmol Rink amide resin (NovaBiochem, San Diego, CA)]. After the removal of the first Fmoc protecting group from the resin with 20% piperidine in N,Ndimethylformamide (DMF) and rinsing of the resin with DMF, the monomer addition cycle was performed by first acetylating the resin with the addition of 1.2 M bromoacetic acid in DMF, followed by N,N-diisopropyl carbodiimide (DIC). The acetylation step was carried out for 45 minutes, and then the resin was washed with DMF. The resin-bound halogen was then displaced by 1.0 M primary amine submonomer in N-methylpyrrolidinone (NMP), which was added to the resin and allowed to react for 90 minutes. The two-step cycle was repeated until the desired length and sequence of the peptoid was obtained, except for the addition of the lysine-like submonomer (NLys), the alkyl submonomers (Nocd), and the proline residue. The displacement step for the Boc-protected NLys submonomer and the Nocd submonomers was extended to 120 minutes, while for the addition of the proline residue, a PyBrop activating system was employed. Additionally, due to poor solubility in NMP, the Nocd submonomer was dissolved at 0.8 M in dichloromethane:methanol (1:1). After the proline addition, the Fmoc group was removed with piperidine as before and the peptoid cycle was continued.

### Example 2

This example illustrates the production of a suitable binding moiety for a binding mimic of the type disclosed herein that is designed to adhere to a coronavirus.

A binding moiety for SARS-CoV-2 coronavirus may be produced using the methodology disclosed in Vanessa Monteil, Hyesoo Kwon, Patricia Prado, Astrid Hagelkrüys, Reiner A. Wimmer, Martin Stahl, Alexandra Leopoldi, Elena Garreta, Carmen Hurtado Del Pozo, Felipe Prosper, J.p. Romero, Gerald Wirnsberger, Haibo Zhang, Arthur S. Slutsky, Ryan Conder, Nuria Montserrat, Ali Mirazimi, Josef M. Penninger. Inhibition of SARS-CoV-2 infections in engineered human tissues using clinical-grade soluble human ACE2. Submitted to Cell, 2020 DOI: 10.1016/j.cell.2020.04.004.

### Example 3

This example illustrates the production of another suitable binding moiety for a coronavirus.

A 23 mer synthetic polypeptide having the amino acid sequence IEEQAKTFLDKFNHEAEDLFYQS, was prepared by automated flow peptide synthesis. The 23 residues selected from the ACE2 α1 helix sequence (IEEQAKTFLDKFNHEAEDLFYQS) showed low fluctuations along the MD simulation trajectory and several important interactions with the spike protein were observed. This was consistent with multiple lines of published data. See R. Yan, Y. Zhang, Y. Li, L. Xia, Y. Guo, and Q. Zhou, Structural basis for the recognition of the SARS-CoV-2 by full-length human ACE2. Science, 2020; and Y. Wan, J. Shang, R. Graham, R.S. Baric, and F. Li, Receptor recognition by novel coronavirus from Wuhan: An analysis based on decade-long structural studies of SARS. J Virol, 2020.

### Example 4

This example illustrates the production of a peptoid-based SP-C binding mimic in accordance with the teachings herein.

A peptoid-based SP-C mimic may be prepared by appending the aforementioned binding agents of EXAMPLES 2 or 3, such as the ACE2 α1 helix sequence (IEEQAKTFLDKFNHEAEDLFYQS), onto the N-terminus of a peptoid-based SP-C mimic, with an intervening short, water-soluble linking sequence. This linking sequence may be between one and ten monomers in length and may be either a peptide sequence (such as the flexible, water-soluble repeating amino acid dimer [Ser-Gly]) or a peptoid sequence, such as an oligo-N-methoxyethylglycine (Nmeg) repeat. Given the limitations of solid-phase peptide and peptoid synthesis, a maximum practical chain length is about 30-32 monomers, and the preferred peptoid-based SP-C mimic (e.g., CLeu3, mono-pCLeu3, or di-pCLeu3) is about 22 N-substituted glycine monomers in length. An additional five water-soluble Nmeg monomers may be added to the amino terminus of the peptoid in the same synthesis, followed by an azide-terminated peptoid submonomer. This peptoid may be HPLC-purified, using methods well known in the art of the preparation of synthetic peptoids, and in particular, using such methods for preparing these SP-C mimetic peptoids. Separately, the above-mentioned ACE2 α1 helix sequence (IEEQAKTFLDKFNHEAEDLFYQS), which binds tightly to the SARS-CoV-2 virus spike protein, may be synthesized (preferably with an alkyne-terminated peptide monomer as the final residue added), and this peptide may also be HPLC-purified. Finally, the purified SP-C mimetic peptoid compound, with its incorporated water-soluble linking moiety and azide terminus, and the ACE2 α1 helix sequence (IEEQAKTFLDKFNHEAEDLFYQS) with its alkyne terminus, are both preferably dissolved in an organic solvent (e.g., dimethylformamide, DMF; or N-methylpyrrolidinone, NMP) and linked utilizing Click chemistry. This chemistry may cause an azide-terminated compound to react specifically and with high yield with an alkyne-terminated compound (See: Jean-François Lutz; Zoya Zarafshani (2008). “Efficient construction of therapeutics, bioconjugates, biomaterials and bioactive surfaces using azide-alkyne “click” chemistry”. Advanced Drug Delivery Reviews. 60 (9): 958-970. doi:10.1016/j.addr.2008.02.004. PMID 18406491). The final conjugate would then be HPLC-purified one more time, and then, in pure form, may be prepared for use.

Various binding moieties may be utilized in the compositions and methodologies disclosed herein. The binding moiety is preferably selected to bind to one or more pathogens or agents of interest. For example, in treating patients infected with the SARS-CoV-2 coronavirus, the binding moiety may be a mimic of the ACE-2 receptor such as, for example, a recombinant ACE-2 protein, which may be, for example, a human recombinant soluble ACE2 (hrsACE2). Preferably, however, the binding moiety is a peptide, and more preferably a peptoid (which may be identified, for example, via binding selection studies), due to the many advantages such smaller molecules provide.

Preferably, the binding moiety has a molecular weight of less than 5000 g/mol, and more preferably has a molecular weight of less than 3200 g/mol. The binding moiety is preferably a peptide with no more than 50 amino acid sequences, and more preferably is a peptoid with no more than 25 amino acid sequences.

Various linking moieties may be utilized in the compositions and methodologies disclosed herein. Preferably, these linking moieties are short, flexible and water soluble. In some embodiments, the linking moiety may be a linking sequence between one and ten monomers in length and may be either a peptide sequence or a peptoid sequence. Specific, non-limiting examples of possible linking moieties include Ser-Gly repeat peptides, oligoethylene glycol (available commercially from Quanta Biosciences), and oligo-N-methoxyglycine peptoid, sometimes referred to as oligo(Nmeg).

The binding mimics disclosed herein may be mixed with various exogeneous surfactants. These include, without limitation, CUROSURF™ intratracheal suspension, which is a sterile, non-pyrogenic pulmonary surfactant intended for intratracheal use. CUROSURF™ is an extract of natural porcine lung surfactant consisting of 99% polar lipids (mainly phospholipids) and 1% hydrophobic low molecular weight proteins (surfactant associated proteins SP-B and SP-C).

These exogeneous surfactants also include INFASURF® intratracheal suspension, a sterile, non-pyrogenic lung surfactant intended for intratracheal instillation. INFASURF® is an extract of natural surfactant from calf lungs which includes phospholipids, neutral lipids, and hydrophobic surfactant-associated proteins B and C (SP-B and SP-C). It is a suspension of calfactant in 0.9% aqueous sodium chloride solution. It has a pH of 5.0 - 6.2 (target pH 5.7). Each milliliter of Infasurf contains 35 mg total phospholipids (including 26 mg phosphatidylcholine of which 16 mg is disaturatedphosphatidylcholine) and 0.7 mg proteins including 0.26 mg of SP-B.

These exogeneous surfactants may also include SURVANTA® (beractant) Intratracheal Suspension, a sterile, non-pyrogenic pulmonary surfactant intended for intratracheal use. SURVANTA® is a natural bovine lung extract containing phospholipids, neutral lipids, fatty acids, and surfactant-associated proteins to which colfosceril palmitate (dipalmitoylphosphatidylcholine), palmitic acid, and tripalmitin are added to standardize the composition and to mimic surface-tension lowering properties of natural lung surfactant. The resulting composition provides 25 mg/mL phospholipids (including 11.0-15.5 mg/mL disaturated phosphatidylcholine), 0.5-1.75 mg/mL triglycerides, 1.4-3.5 mg/mL free fatty acids, and less than 1.0 mg/mL protein. It is suspended in 0.9% sodium chloride solution, and heat-sterilized. Its protein content consists of two hydrophobic, low molecular weight, surfactant-associated proteins commonly known as SP-B and SP-C. It does not contain the hydrophilic, large molecular weight surfactant-associated protein known as SP-A. Each mL of SURVANTA contains 25 mg of phospholipids.

These exogeneous surfactants may also include other synthetic pulmonary surfactants such as, for example, Colfosceril palmitate (Exosurf), a mixture of DPPC with hexadecanol and tyloxapol added as spreading agents; Pumactant (Artificial Lung Expanding Compound or ALEC), a mixture of DPPC and PG; KL-4, which consists of DPPC, palmitoyl-oleoyl phosphatidylglycerol, and palmitic acid, combined with a 21 amino acid synthetic peptide that mimics the structural characteristics of SP-B; Venticute, which contains DPPC, PG, palmitic acid and recombinant SP-C; and Lucinactant, which contains DPPC, POPG, and palmitic acid.

In some embodiments, the binding mimics disclosed herein may be spiked into exogenous surfactants that also contain a plurality of (preferably SP-C) mimics that are not engineered to bind to viruses and other pathogens. In such embodiments, the binding and non-binding mimics may be present in various ratios to achieve desired effects.

The binding mimics disclosed herein may be based on peptide analogs of SP-C (e.g., with all-leucine substitution into hydrophobic helical region, as first demonstrated by Dr. Jan Johansson, see: Brown NJ, Johansson J, Barron AE, “Biomimicry of surfactant protein C,” Acc. Chem. Res. 2008, 41, 1409-1417.); peptoid analogs of SP-C; or hybrid peptide/peptoid molecules (preferably with just two peptoid residues at positions 5 and 6, using octadecylamine peptoid monomers to replace the two thioester-linked palmitoyl chains).

While the binding mimics disclosed herein are especially suitable for use in the lungs, they may be used to treat infections in other parts of the body as well. For example, SP-C also occurs in the Eustachian tube of the ear. Hence, the binding mimics disclosed herein may be especially suitable for treating various ear infections such as, for example, otitis media, chronic suppurative otitis media, and otitis externa. In such uses, the binding moiety may be tailored for the relevant pathogens. For example, the majority of cases of swimmer’s ear are due to infection by Pseudomonas aeruginosa and Staphylococcus aureus, followed by a great number of other gram-positive and gram-negative species. Candida albicans and Aspergillus species (such as Aspergillus ƒumigatus) are the most common fungal pathogens responsible for the condition. Hence, compositions and methodologies may be used in accordance with the teachings herein to treat these conditions using binding mimics (or mixtures of binding mimics) equipped with one or more binding moieties that target one or more of these pathogens. In the case of Pseudomonas aeruginosa, for example, the binding moiety may be a mimic of one or more laminins. In the case of Staphylococcus aureus, the binding moiety may be a mimic of one or more components of the host ECM such as collagen, fibrinogen, or Fn, or a mimic of a glycoprotein such as von Willebrand factor (vWF), or a mimic of immunoglobulin (or the Fc region of the antibody or the Fab regions of the B-cell receptor), or a mimic of one or more portions of the foregoing. In the case of Candida albicans, the binding moiety may be a mimic of mucin or a component thereof such as, for example, the 66-kDa cleavage product of the 118-kDa C-terminal glycopeptide of mucin, or may be a mimic of a complement control protein such as Factor H (FH) or a portion thereof. In the case of Aspergillus ƒumigatus, the binding moiety may be a mimic of fibrinogen C domain-containing protein 1 (FIBCD1) or a portion thereof, or a mimic of an MBL-associated serine protease (MASP) such as MASP-1, MASP-3 or a portion thereof.

In some applications, antibiotic or antifungal agents may be added to surfactant formulations containing the binding mimics disclosed herein. In some cases, these antibiotic or antifungal agents may act synergistically with the binding mimic (for example, by inactivating pathogens while they are bound by the binding mimic). For instance, antimicrobial peptoids, antimicrobial peptides, and antibiotics (such as, for example, Tobramycin, Ofloxacin, or Azithromycin) may be added to surfactant formulations containing the binding mimics disclosed herein. The binding mimics disclosed herein may be utilized in conjunction with the peptoids disclosed in U.S. 8,445,632 (Barron et al.), entitled “Selective Poly-N-Substituted Glycine Antibiotics”, which is incorporated herein by reference in its entirety, the peptoids disclosed in Diamond G, Molchanova N., Herlan C., Fortkort J.A., Lin J.S., Figgins E., Bopp N., Ryan L.K., Chung D., Adcock R.S., Sherman M., Barron A.E. Potent Antiviral Activity against HSV-1 and SARS-CoV-2 by Antimicrobial Peptoids. Pharmaceuticals (Basel). 2021 Mar 31;14(4):304. doi: 10.3390/ph14040304. PMID: 33807248; PMCID: PMC8066833, which is incorporated herein by reference in its entirety) and in halogenated derivatives of such peptoids. The binding mimics disclosed herein may also be utilized in conjunction with the peptoids disclosed in U.S. 9,938,321 (Kirshenbaum et al.), U.S. 9,315,548 (Kirshenbaum et al.) and U.S. 8,828,413 (Kirshenbaum et al.), all of which are incorporated herein by reference in their entirety. The binding mimics disclosed herein may also be utilized in conjunction with halogenated analogs of the peptoids of Barron et al. and Kirshenbaum et al. These halogen analogs may feature halogen substitution on one or more of the side chain or ring structures by one or more halogens, and preferably include bromo-substituted or chloro-substituted analogs.

In some applications of the compositions and methodologies disclosed herein, the binding mimics may be applied as aerosolized powders. Methods for making and applying such powders are described, for example, in Daniher, D., Mccaig, L., Ye, Y., Veldhuizen, R., Lewis, J., Ma, Y., & Zhu, J. (2020). Protective effects of aerosolized pulmonary surfactant powder in a model of ventilator-induced lung injury. International Journal of Pharmaceutics, 583, 119359. doi:10.1016/j.ijpharm.2020.119359, which is incorporated herein by reference in its entirety.

In some embodiments, the binding mimics may also be utilized in conjunction with suitable surfactant lipids and analogs thereof. These include, without limitation, the lipids disclosed in U.S. 10,532,066 (Voelker et al.), entitled “Surfactant Lipids, Compositions Thereof, And Uses Thereof”, which is incorporated herein in its entirety; and the lipids disclosed in U.S. 2020/0009165 (Voelker), entitled “Methods And Compositions For Treating And Preventing Respiratory Related Diseases And Conditions With Xylitol-Headgroup Lipid Analogs”, which is incorporated herein in its entirety.

The above description of the present invention is illustrative and is not intended to be limiting. It will thus be appreciated that various additions, substitutions and modifications may be made to the above-described embodiments without departing from the scope of the present invention. Accordingly, the scope of the present invention should be construed in reference to the appended claims.

What is claimed is:

1. A method for treating an individual for an infection caused by a pathogen, the method comprising:
administering to an individual who has said infection or who is at risk of developing said infection, an amount of at least one composition, wherein the amount of the composition is effective to inhibit said infection, and wherein the composition includes
(a) a hydrophobic, helical region,
(b) an N-terminal region that includes at least one proline residue,
(c) a first linking moiety that links the hydrophobic helical region to the N-terminal region, said linking moiety comprising at least one lysine-like side chain,
(d) a binding moiety which binds to the pathogen, and
(e) a second linking moiety that links the binding moiety to the N-terminal region.

2. The method of claim 1, wherein the second linking moiety links the binding moiety to a proline residue in the N-terminal region.

3. The method of claim 1, wherein the second linking moiety is hydrophilic.

4. The method of claim 1, wherein the N-terminal region includes at least one alkyl chain.

5. The method of claim 1, wherein the N-terminal region includes at least one C-18 alkyl chain.

6. The method of claim 1, wherein the N-terminal region includes a plurality of C-18 alkyl chains.

7. The method of claim 1, wherein the composition is a poly-A-substituted glycine.

8. The method of claim 1, wherein the composition has the formula H-NpmNpm(Pro-L-A)NValNpmNLeuNLysNLys(NSpe)₁₄-NH₂.

9. The method of claim 1, wherein the composition has the formula H-NpmNpm(Pro-L-A)NValNpmNLeuNLysNLys(NSpeNSpeNssb)₄-NSpeNSpe-NH₂.

10. The method of claim 1, wherein the composition has the formula H-NpmNpm(Pro-L-A)NValNpmNLeuNLysNLys(NSpeNssbNssb)₄NSpeNSpe-NH₂.

11. The method of claim 1, wherein the composition has the formula H-NpmNpm(Pro-L-A)NValNpmNLeuNLysNLys(NSpeNssbNssb)₄NSpe-NH₂.

B1-D37. (canceled)
